(19) United States
(12) Patent Application Publication
(10) Pub. No.: US 2012/0226126 A1
(43) Pub. Date: Sep. 6, 2012

(54) Method and Apparatus for Acquiring Data Relating to a Physiological Condition of a Subject When Chest Wall Access is Limited

(75) Inventors: Edward Busse, Vancouver (CA); James Alexander Burns, Vancouver (CA); David MacQuarrie, Vancouver (CA)

(73) Assignee: Heart Force Medical Inc., Vancouver, British Columbia (CA)

(21) Appl. No.: 13/057,980

(22) PCT Filed: Aug. 7, 2009
(86) PCT No.: PCT/CA2009/001111

§ 371 Date: Oct. 10, 2011

Related U.S. Application Data
(60) Provisional application No. 61/087,123, filed on Aug. 7, 2008.

(51) Int. Cl. A61B 5/11; A61B 5/042; A61B 5/024

(52) U.S. Cl. 600/380; 600/527

(57) ABSTRACT

An apparatus for acquiring and outputting data relating to a physiological condition of a subject, the apparatus including: a sensor device including an accelerometer provided in a tube for insertion into an esophagus of said subject, the sensor device for detecting, converting and transmitting digital signals corresponding to analog ballistocardiograph signals; and a computer including a processor in communication with the sensor device, the computer for receiving the digital signals from the sensor device and generating and outputting a report relating to the physiological condition of the subject.

### RELATED APPLICATION

This application claims benefit under 35 U.S.C. 119(e) of U.S. Provisional Patent Application Ser. No. 61/087,123, filed Aug. 7, 2008, entitled “Trans-Esophageal Monitoring System and Apparatus for Monitoring, Acquisition and Transmission of Disparate Cardiovascular Signals”, which is incorporated herein by reference in its entirely.

### TECHNICAL FIELD

The present invention relates to a method and apparatus for acquiring data relating to a physiological condition of a subject in applications in which access to a chest wall of the subject is limited.

### BACKGROUND

Advancements in medicine continue to improve the diagnosis and treatment of various heart conditions. Analysis of the electrical and mechanical activity of the heart provided by electrocardiograph (ECG) and ballistocardiograph (BCG) waveforms, respectively, is one diagnostic method. FIGS. 1(a) and 1(b) show the relationship between rhythmic electrical functions and related physical motions of a heart in which FIG. 1(a) is a sample ECG waveform and FIG. 1(b) is a sample BCG waveform.

An ECG waveform is typically obtained by detecting electrical activity of the heart through a chest wall of the subject. In general, ECG waveforms provide a static record of a subject's cardiovascular function at the time of testing. Abnormal patterns on an ECG may be non-specific, and therefore observed with a variety of different conditions. They may even be a normal variant and not reflect any abnormality at all.

A BCG waveform is typically obtained using an apparatus that includes a low-friction table and an accelerometer, which transduces the motion of the entire table caused by the systolic ejection of a heart of a subject lying on the table. Subjects are often placed in the prone position on the apparatus.

BCG waveforms either alone or in combination with ECG waveforms provide useful diagnostic information. An improved apparatus for obtaining BCG data and/or ECG data in applications in which access to the chest wall is limited is desirable.

### SUMMARY

In one aspect of the invention there is provided an apparatus for acquiring and outputting data relating to a physiological condition of a subject, the apparatus including: a sensor device including an accelerometer provided in a tube for insertion into an esophagus of the subject, the sensor device for detecting, converting and transmitting digital signals corresponding to analog ballistocardiograph signals; and a computer including a processor in communication with the sensor device, the computer for receiving the digital signals from the sensor device and generating and outputting a report relating to the physiological condition of the subject.

In another aspect of the invention there is provided a method for acquiring and outputting data relating to a physiological condition of a subject, the method including: detecting analog signals using a sensor device having a three-axis accelerometer, the analog signals being ballistocardiograph signals, the three-axis accelerometer device being provided in a tube for insertion in an esophagus of the subject; converting the analog signals into digital signals; transmitting the digital signals to a computer; performing an analysis of the digital signals; and generating and outputting a report relating to the physiological condition.

In yet another aspect of the invention there is provided a sensor device for use in an apparatus for acquiring and outputting data relating to a physiological condition of a subject, the sensor device including: a three-axis accelerometer received in a tube for insertion into an esophagus of the subject, the three-axis accelerometer for sensing vibrations of a wall the esophagus; an analog to digital converter provided in communication with the three-axis accelerometer, the analog to digital converter for receiving three analog signals and converting the three separate analog signals into digital signals, the three analog signals being ballistocardiograph signals corresponding to each axis of the three-axis accelerometer; and a power source in communication with the three-axis accelerometer and the analog to digital converter.

### DRAWINGS

The following figures set forth embodiments of the invention in which like reference numerals denote like parts. Embodiments of the invention are illustrated by way of example and not by way of limitation in the accompanying figures.

FIG. 1(a) is an example of an electrocardiogram waveform;

FIG. 1(b) is an example of a ballistocardiogram waveform;

FIG. 2 is a schematic diagram of an apparatus for acquiring and outputting data relating to a physiological condition of a subject according to an embodiment;

FIG. 3a is a schematic side view of a portion of a tube of the apparatus of FIG. 2 according to an embodiment;

FIG. 3b is a schematic side view of a portion of a tube of the apparatus of FIG. 2 according to another embodiment;

FIG. 4 is a block diagram of selected components of the sensor device of the apparatus of FIG. 2;

FIG. 5 is a schematic view of portions an apparatus for acquiring and outputting data relating to a physiological condition of a subject according another embodiment;

FIG. 6 is a flowchart depicting a method of operation of the apparatus of FIG. 2 according to an embodiment;

FIG. 7 is a schematic diagram of an apparatus for acquiring and outputting data relating to a physiological condition of a subject according to another embodiment;

FIG. 8 is a block diagram of selected components of the sensor device of the apparatus of FIG. 7;

FIG. 9 is a flowchart depicting a method of operation of the apparatus of FIG. 6 according to an embodiment;

FIG. 10 is a schematic diagram of an apparatus for acquiring and outputting data relating to a physiological condition of a subject according to another embodiment; and

FIG. 11 is a schematic view of an application of an apparatus for acquiring and outputting data relating to a physiological condition of a subject in a critical care unit.

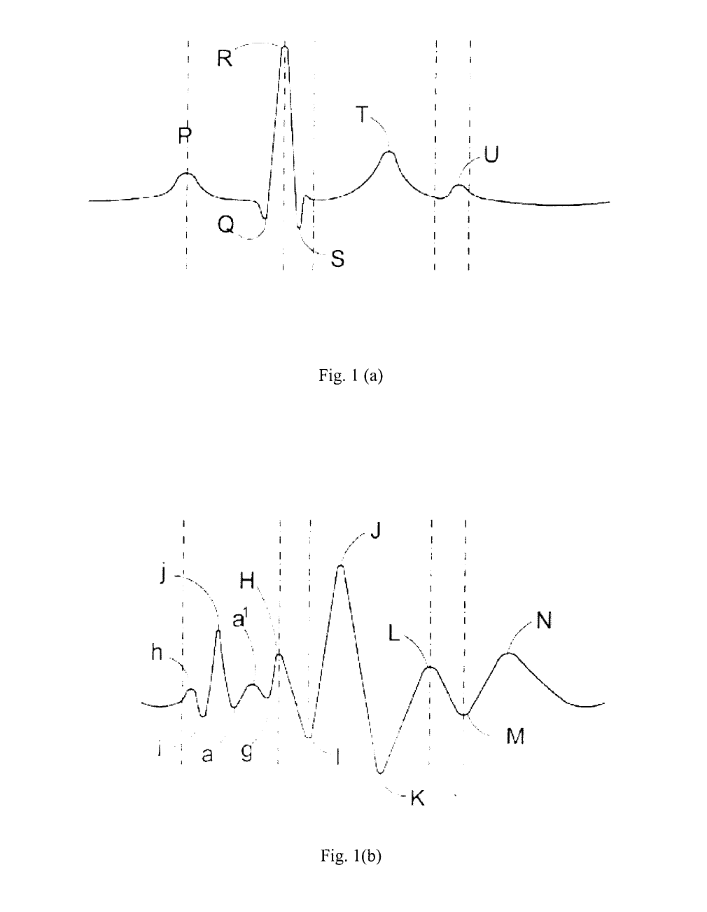

### DETAILED DESCRIPTION OF EMBODIMENTS OF THE INVENTION

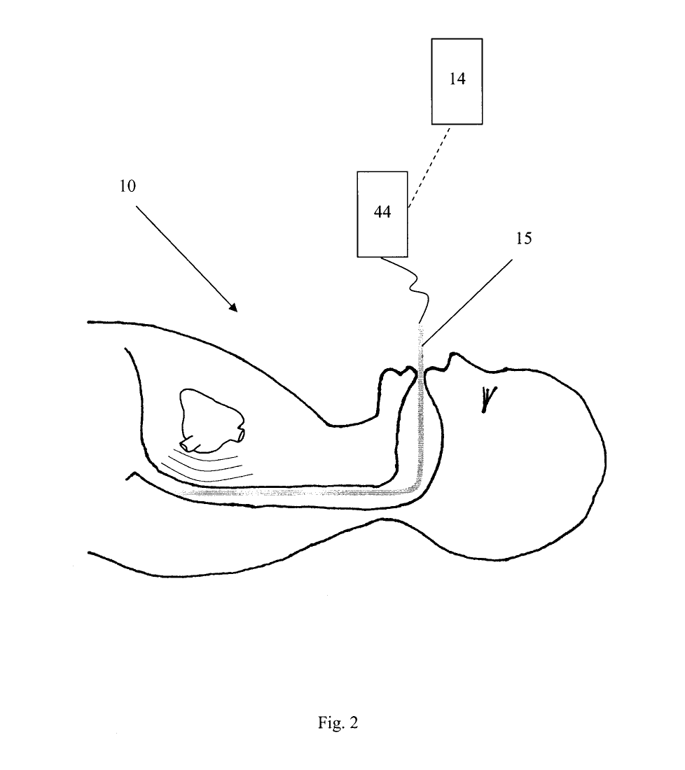

Referring to FIG. 2, an apparatus 10 for acquiring data relating to a physiological condition of a subject is generally shown. The apparatus 10 includes a sensor device 12 that is in communication with a computer 14. The sensor device 12 includes an accelerometer (not shown) that is in communication with a transceiver 44 via wire 28. The accelerometer is received in a tube 15 and is provided for detecting, analog ballistocardiograph (BCG) signals. The transceiver 44 receives the analog BCG signals from the accelerometer, converts the signals and transmits digital signals corresponding to analog BCG signals to the computer 14. The computer 14 receives the digital BCG signals and performs at least one of: outputting a visual representation of the digital signals in the form of a BCG waveform and analyzing the digital signals in order to output a report relating to the physiological condition of the subject.

The computer 14 includes a radio device (not shown), a user interface (not shown), a processor (not shown) and a computer memory (not shown) that stores software that is executable by the processor. The software may alternatively be stored on another type of computer readable medium. The computer 14 controls the sensor device 12 by sending commands wirelessly via the radio device in order to initiate and terminate detection and transmission of the BCG signals. The computer receives the digital BCG signals wirelessly from the transceiver 44 of the sensor device 12. Software is provided for at least one of: outputting a visual representation of the digital signals in the form of a BCG waveform and analyzing the digital signals in order to output a report relating to the physiological condition of the subject. The BCG waveform and information corresponding to the digital signals may be printed by a printer (not shown) that is in communication with the computer 14 or may be displayed by a monitor (not shown) that is in communication with the computer 14.

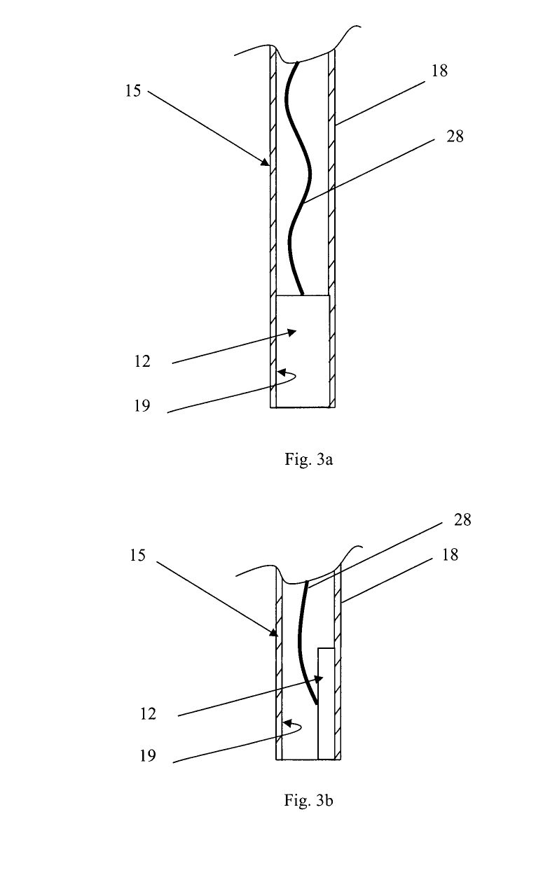

Referring also to FIG. 3a, the accelerometer is provided in a housing 16 that is sized to be received in the tube 15 and is connected to the transceiver 44 by wires 28. The wires 28 extend through the tube 15 and exit the tube 15 outside of the subject's body. A length of the tube 15 is sufficient to allow the tube 15 to be inserted through a subject's nose or mouth and reach a location in the esophagus that is adjacent to the heart. An outer surface 18 of the tube 15 is marked with distance measurements (not shown) in order aid in location of the accelerometer adjacent to the heart. The distance measurements allow the operator to determine the distance that the accelerometer has traveled into the chest during insertion. Other markings may also be provided on the tube 15 to facilitate correct insertion thereof. The tube 15 is typically inserted by a skilled technician to ensure that the BCG signals transmitted by the accelerometer provide reliable information.

As will be understood by a person skilled in the art, the tube 15 is flexible and made of a biocompatible material, such as silastic™ or Tygon™, for example. An outer diameter of the tube 15 is generally less than 1.5 cm. In one embodiment, the outer diameter of the tube 15 is between 1.0 cm and 1.4 cm.

As shown, the housing 16 is received in a distal end portion 18 of the tube 15 and is sized to fit snugly within the tube 15. An adhesive may also be used to secure the housing 16 to the inner surface 19 of the tube 15 or, alternatively, the housing 16 may not be secured to the tube 15. The housing 16 is made of a suitable biocompatible material and may be provided by the accelerometer manufacturer or may be a separate component. A person skilled in the art will appreciate that any suitable manner of securing the housing 16 within the tube 15 may be employed.

Referring to FIG. 3b, another embodiment of a sensor device 12 for use in the apparatus 10 of FIG. 2 is generally shown. In this embodiment, rather than being cylindrical in shape, the housing 16 for the accelerometer is block-shaped and adhered to an inner surface 19 of the tube 15.

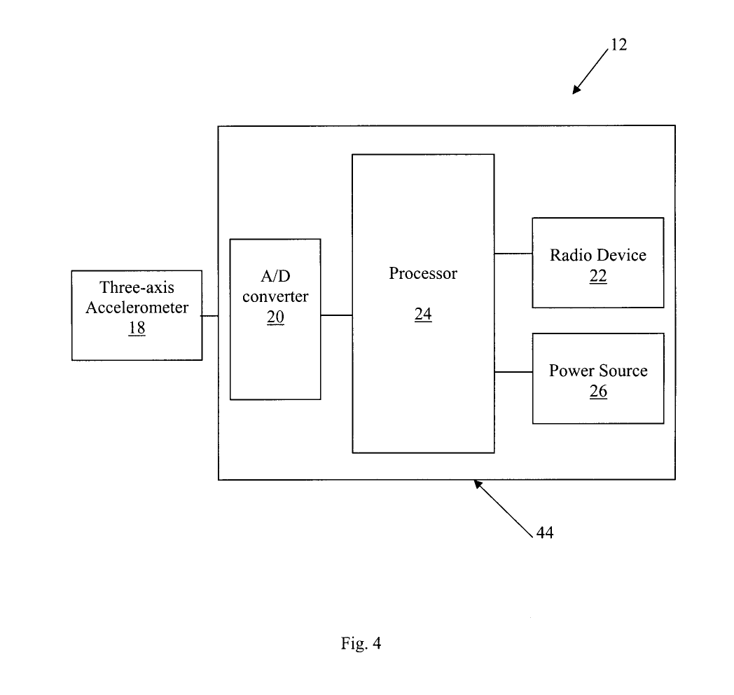

Referring to FIG. 4, the accelerometer of the sensor device 12 is a three-axis accelerometer 18 for sensing vibrations of the esophagus wall of the subject. The three-axis accelerometer 18 senses the mechanical motion of the esophagus wall caused by heart movement in three axes: x, y and z and outputs three separate BCG signals that correspond to the x, y and z axes. An example of a three-axis accelerometer that is suitable for use in the sensor device 12 is a LIS3L02AL MEMS Inertial sensor, which is manufactured by ST Microelectronics.

The transceiver 44 of the sensor device 12 includes an analog to digital converter 20 that receives the three separate analog ballistocardiograph signals corresponding to each axis of the three-axis accelerometer 18 and converts the three separate analog signals into digital signals. The BCG signals are amplified by amplifiers set to appropriate gain levels and band-limited by linear filtering prior to being sampled by the analog-to-digital converter 20. Suitable analog-to-digital converters include 8, 12, 16, 24 and 32 bit analog-to-digital converters having a sample rate of 500 samples per second, for example.

The transceiver 44 further includes a radio device 22 for transmitting the digital signals to the computer 14, a processor 24 and a power source 26. The components of the transceiver 44 are mounted in a transceiver housing. The processor 24 communicates with each of the electronic components of the transceiver 44 and generally controls operation thereof. The transceiver 44 further includes a non-volatile memory (not shown) that is programmed with accelerometer calibration data.

Commands for initiating and terminating operation of the accelerometer 18 are received from the computer 14 via the radio device 22. The radio device 22 may be any device that is capable of wireless communication. In one embodiment, the radio device 22 is a Bluetooth™ communication device capable of short range wireless communication.

The power source 26 is generally a battery capable of providing sufficient power to operate the sensor device 12. The power source 16 may have a finite life, or alternatively, may be rechargeable.

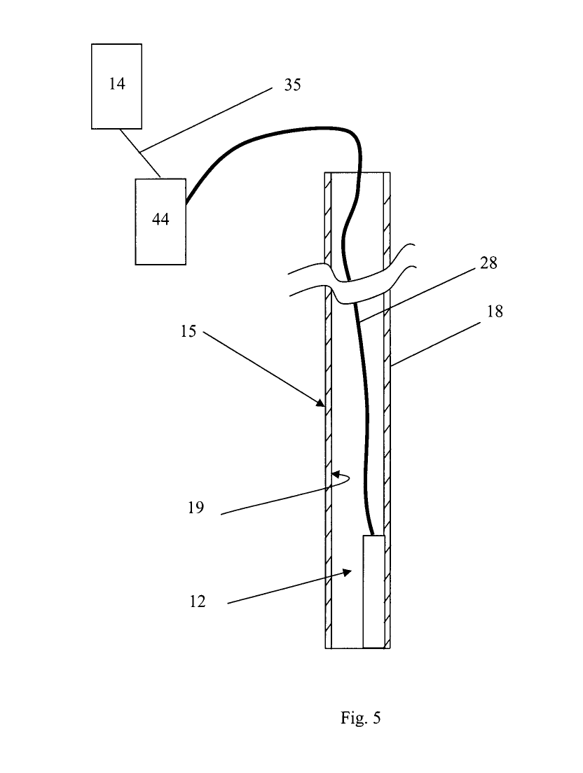

In another embodiment, which is shown in FIG. 5, the sensor device 12 and the computer 14 are connected by wires 35. In this embodiment, the radio device 22 may be omitted from the sensor device 12.

In another embodiment, the accelerometer 18 is received in a recess (not shown) that is provided in a wall of the tube 15. Once the accelerometer 18 is in place, the recess is covered with a layer of a suitable material to fix the accelerometer 18 in place within the wall of the tube 15.

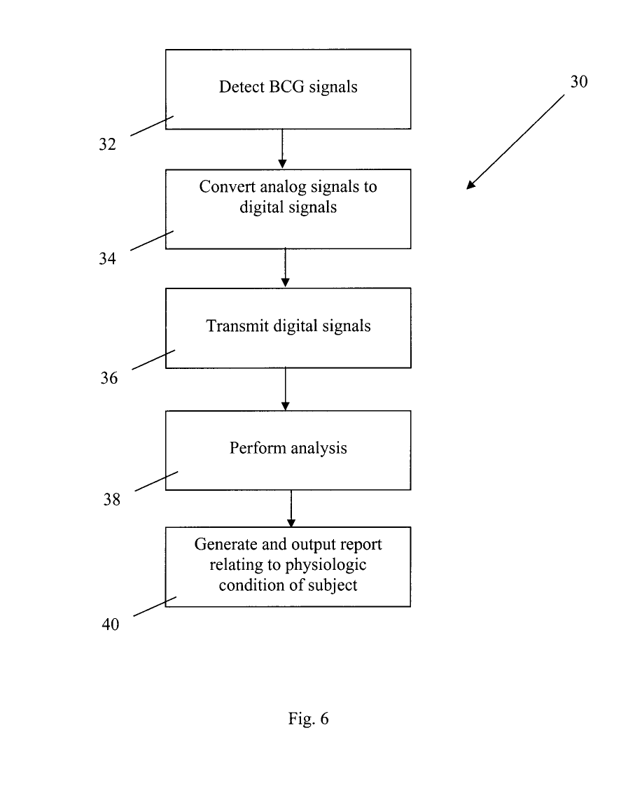

Operation of the apparatus for acquiring and outputting data relating to a physiological condition of a subject according to another embodiment will now be described with reference to FIG. 6.

The method 30 is executed once for each test that is performed on a subject. At step 32, the BCG signals are detected by the sensor device 12. In order to detect the signals, the tube 15 housing the accelerometer 18 is first inserted through the nose and throat into the esophagus of the subject. This is typically done when a local anesthetic has been applied to the back of the throat to suppress the gag reflex. The subject may or may not be sedated during insertion of the tube 15. The distance measurements on the outer wall 18 of the tube 15 are used in order to determine when the accelerometer 18 has reached a location that is adjacent to the heart. When properly inserted, the tube 15 is in contact with the esophagus wall, however, as will be appreciated by a person skilled in the art, the sensor device 12 will still detect and transmit BCG signals if the tube 15 is not in contact with the esophagus wall.

Detection of the signals is initiated by a ‘start’ command that is received by the sensor device 12 and detection continues until an ‘end’ command is received. The command may be issued by pressing a designated key on the computer 14 that is in communication with the sensor device 12. The same key, or a different key, is then pressed in order to send the “end” command to the sensor device 12 upon completion of the test.

As the signals are detected, they are amplified and converted to digital signals in real time, as indicated at step 34. Once converted, the digital signals are transmitted to the computer 14, as indicated at step 36. Once the digital signals are received by the computer 14, an analysis of the BCG data is performed, as indicated at step 38. At step 40, a report relating to the physiological condition of a subject is generated and output by the computer 14.

The report that is generated by the computer 14 may take a number of different forms depending on the particular application. The report may be a BCG waveform corresponding to the digital signals that is displayed on a hospital monitor, for example. The reports may be customized to provide only the information that is desired for each application. The report may be printed or displayed by the computer. Other methods for outputting the report may also be provided.

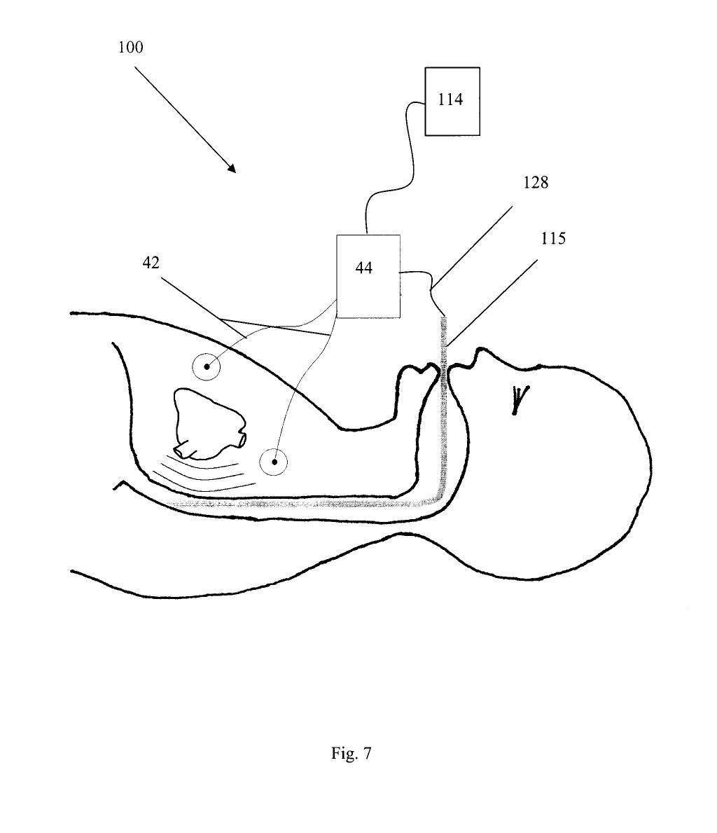

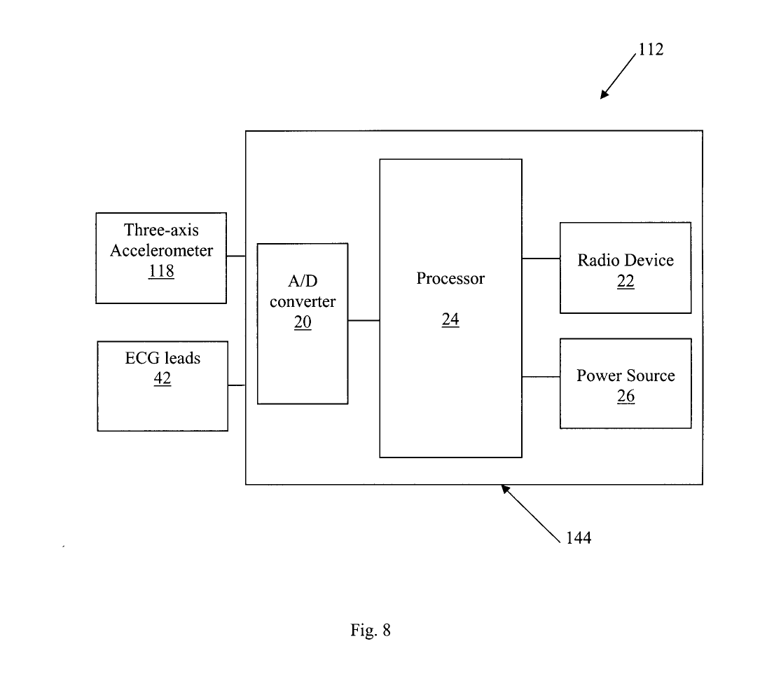

Referring to FIGS. 7 and 8, another apparatus 100 for acquiring data relating to a physiological condition of a subject is generally shown. The apparatus 100 includes a tube 115 for insertion in an esophagus of a subject, a sensor device 112 including an accelerometer 18, a transceiver 144 and a pair of electrocardiograph (ECG) leads 42 and a computer 114 in communication with the sensor device 112. The sensor device 112 is provided for detecting, converting and transmitting digital signals corresponding to analog BCG signals and analog ECG signals. The computer 114 communicates with the transceiver 144 of the sensor device 112 via wires 135. The ECG leads 42 are coupled to the subject via electrodes to detect and transmit an analog ECG signal to the transceiver 44. The transceiver 44 amplifies and converts the analog ECG and BCG signals to digital signals and transmits the digital signals to the computer 114. The computer 114 receives the synchronized digital ECG and BCG signals and performs at least one of: outputting a visual representation of the digital signals in the form of a BCG waveform and analyzing the digital signals in order to output a report relating to the physiological condition of the subject.

It will be appreciated by a person skilled in the art that although a wired connection has been described, the transceiver 144 and computer 114 may communicate wirelessly.

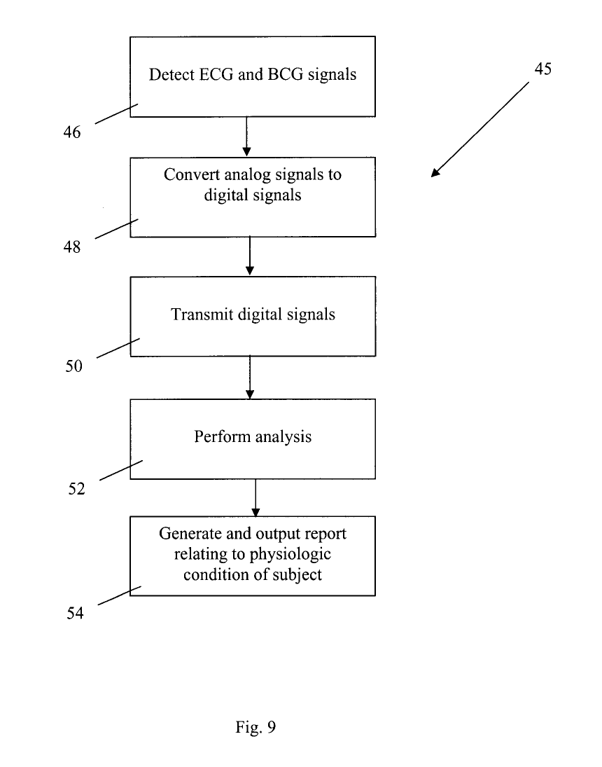

Operation of the apparatus 100 for acquiring and outputting data relating to a physiological condition of a subject according to another embodiment will now be described with reference to FIG. 9. The method 45 is executed once for each test that is performed on a subject. At step 46, the ECG and BCG signals are detected by the sensor device 112. In order to detect the signals, the tube 115 housing the accelerometer 118 is first inserted through the nose and throat into the esophagus of the subject, in a manner that has been previously described, and the ECG leads 42 are coupled via electrodes to the subject's chest wall.

A ‘start’ command initiates detection of the ECG and BCG signals and signal detection continues until an ‘end’ command is received. The ‘start’ command is issued by pressing a designated key on the computer 114 that is in communication with the sensor device 112. The same key, or a different key, is then pressed in order to send the “end” command upon completion of the test.

As the BCG and ECG signals are detected, they are amplified and converted to digital signals in real time by the transceiver 144, as indicated at step 48. Once converted, the digital signals are transmitted to the computer 114, as indicated at step 50. Once the digital signals are received by the computer 114, an analysis of the ECG and BCG data is performed, as indicated at step 52. At step 54, a report relating to the physiological condition of a subject is generated and output by the computer 114.

The report that is generated by the computer 114 may take a number of different forms depending on the particular application. The report may be a synchronized ECG-BCG waveform set corresponding to the digital signals that is displayed on a hospital monitor, for example. The report may alternatively provide information determined by performing analysis on the ECG and BCG data. Methods for analyzing a synchronized ECG-BCG waveform set are described in PCT Publication Nos. WO/2009/073986 and WO/2009/073982, which are herein incorporated by reference.

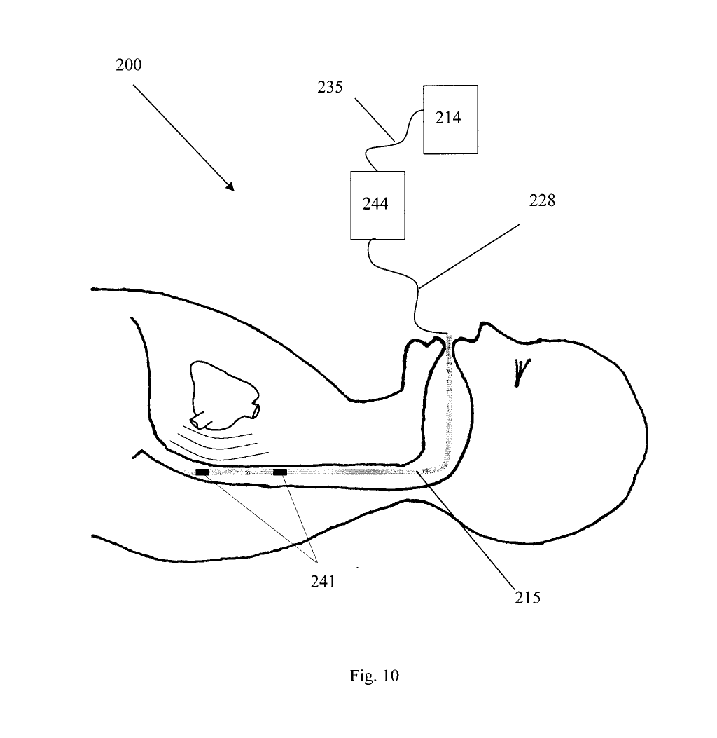

Referring to FIG. 10, another apparatus 200 for acquiring data relating to a physiological condition of a subject is generally shown. The apparatus 200 includes a sensor device for detecting, converting and transmitting digital signals corresponding to analog ECG and BCG signals to a computer 214. The sensor device includes an accelerometer (not shown) and a pair of conductive strips 241 that are coupled to tube 215 for insertion in an esophagus of a subject and a transceiver 244. In the embodiment shown in FIG. 10, the conductive strips 241 are spaced from one another and coupled to an outer surface of the tube 215. The conductive strips 241 are coupled to ECG leads (not shown), which communicate with the transceiver 244, to allow for detection of the electrical activity of the heart. The accelerometer and ECG leads communicate with transceiver 244 via wires 228 and the computer 214 communicates with the transceiver 244 via wires 235 or wirelessly. The transceiver 244 receives the analog ECG and BCG signals and converts and transmits corresponding digital signals to the computer 214. The computer 214 receives the synchronized pair of digital ECG signal and the digital BCG signals and performs at least one of: outputting a visual representation of the digital signals in the form of an ECG-BCG waveform and analyzing the digital signals in order to output a report relating to the physiological condition of the subject. Operation of the Apparatus 200 is similar to operation of the apparatus 100 and therefore will not be repeated.

While detection of BCG signals is possible when the tube 215 is not in contact with the esophagus wall, detection of ECG signals is not. It will be appreciated by a person skilled in the art that because the esophagus is made up of flexible, soft tissue, a skilled technician is able to consistently insert the tube 215 so that the conductive strips are in contact with the esophagus wall.

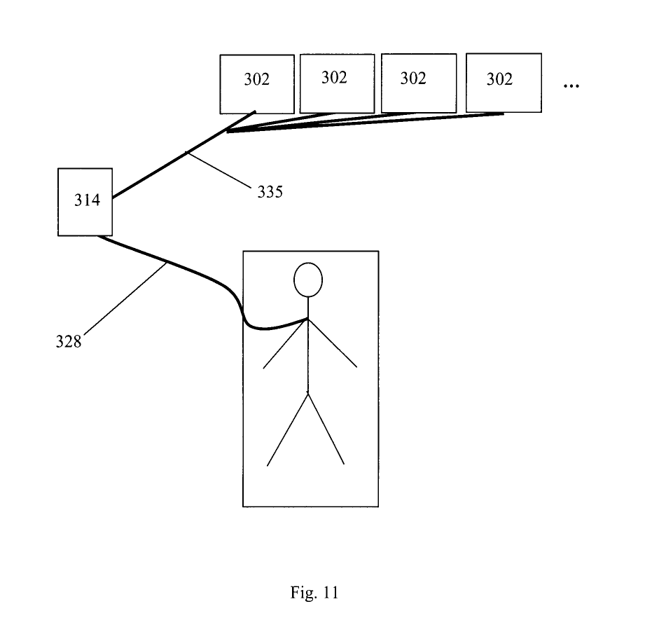

Referring to FIG. 11, an application of apparatus 10, 100, 200 is generally shown. In this application, the apparatus 10, 100, 200 is incorporated into a critical care system of a hospital. Critical care systems typically include a plurality of devices (not shown) that are maintained in contact with a patient for acquiring different types of physiological data including: blood pressure, heart rate, blood oxygenation levels and venous pressures. The devices communicate with a computer 314, which outputs relevant physiological information of the patient in real time to monitors 302. This allows medical personnel to closely monitor the patient's condition. It will be appreciated by a person skilled in the art that although only four monitors are shown, there may be any number of monitors. In addition, one monitor may be used to output physiological information relating to more than one of the devices.

In the application of FIG. 10, the sensor device 12, 112, 212 is one of the devices that is maintained in contact with the patient. The tube 15, 115, 215 is inserted into the patient's esophagus in order to transmit BCG signals to the computer 314. As has been described, ECG signals will also be transmitted when apparatus 100 and 200 are used. Software is stored on the computer 314 for transforming the digital signals into relevant output, such as a BCG waveform, for example, for display on the monitors 302. The relevant output would be continuously updated in order to provide real time information about the patient. For apparatus 10, which does not include ECG leads 42, 242, an ECG may be provided separately as one of the plurality of devices of the critical care system.

The computer 314 communicates with the sensor device 12, 112, 212 other devices and monitors 302 via wires 328 and 335, respectively, as shown. In some critical care units, communication may be wireless, however, wired communication is often preferred in such environments.

The forces generated by movement of the heart tend to be stronger in the esophagus than on the chest wall. Therefore, a sensor device that detects the forces via the esophagus is likely to provide a more accurate representation of the subject's heart forces.

The sensor devices described herein are trans-esophageal sensor devices that are useful in applications in which BCG data and/or ECG is desirable but access to the chest wall is limited or not available. Such applications include: during heart surgery, recovery from heart surgery, during major abdominal or thoracic surgical procedures or when a patient has undergone chest trauma, for example.

In addition, as people age, the stiffness of their ribs gradually increases. As a result, conduction of mechanical heart forces through the chest wall becomes less effective and there is deterioration in the quality of data that is produced from chest-mounted sensor devices. A sensor device that detects the forces via the esophagus is not affected by aging of the subjects.

Specific embodiments have been shown and described herein. However, modifications and variations may occur to those skilled in the art. All such modifications and variations are believed to be within the scope and sphere of the present invention.

1. An apparatus for acquiring and outputting data relating to a physiological condition of a subject, said apparatus comprising:
a sensor device comprising an accelerometer provided in a tube for insertion into an esophagus of said subject, said sensor device for detecting, converting and transmitting digital signals corresponding to analog ballistocardiograph signals; and
a computer comprising a processor in communication with said sensor device, said computer for receiving said digital signals from said sensor device and generating and outputting a report relating to said physiological condition of said subject.

2. An apparatus as claimed in claim 1, wherein said computer communicates with said sensor device to initiate and terminate detection of said analog ballistocardiograph signals.

3. An apparatus as claimed in claim 2, wherein said computer communicates with said sensor device via a wireless connection.

4. An apparatus as claimed in claim 1, wherein said computer includes software executable by said processor for analyzing and ballistocardiograph data corresponding to said digital signals.

5. An apparatus as claimed in claim 1, wherein said report is displayed on a monitor.

6. An apparatus as claimed in claim 1, wherein said sensor device comprises a pair of electrocardiograph leads, said sensor device detecting, converting and transmitting digital signals corresponding to analog electrocardiograph signals.

7. A method for acquiring and outputting data relating to a physiological condition of a subject, said method comprising:
detecting analog signals using a sensor device having a three-axis accelerometer, said analog signals being ballistocardiograph signals, said three-axis accelerometer device being provided in a tube for insertion in an esophagus of said subject;
converting said analog signals into digital signals;
transmitting said digital signals to a computer;
performing an analysis of said digital signals; and
generating and outputting a report relating to said physiological condition.

8. A method as claimed in claim 7, wherein said computer communicates with said sensor device to initiate and terminate detection of said analog signals.

9. A sensor device for use in an apparatus for acquiring and outputting data relating to a physiological condition of a subject, said sensor device comprising:
a three-axis accelerometer received in a tube for insertion into an esophagus of said subject, said three-axis accelerometer for sensing vibrations of a wall said esophagus;
an analog to digital converter provided in communication with said three-axis accelerometer, said analog to digital converter for receiving three analog signals and converting said three separate analog signals into digital signals, said three analog signals being ballistocardiograph signals corresponding to each axis of said three-axis accelerometer; and
a power source in communication with said three-axis accelerometer and said analog to digital converter.

10. A sensor device as claimed in claim 9, comprising a radio device provided in communication with said analog to digital converter and said power source for transmitting said digital signals to a computer.

11. A sensor device as claimed in claim 9, comprising a pair of conductive strips coupled to an outer surface of said tube, said conductive strips being provided at a distal end of electrocardiograph leads and for coupling to said subject via electrodes for detecting an analog electrocardiograph signal, said analog electrocardiograph signal being converted to a digital signal by said analog to digital converter.
